(19) United States
(12) Patent Application Publication
(10) Pub. No.: US 2026/0307082 A1
(43) Pub. Date: Oct. 8, 2026

(54) HYBRID FILTER UNIT

(71) Applicant: BRAIRTECH SWEDEN AB, MALMÖ (SE)

(72) Inventor: Behzad FARZINPOUR, MALMÖ (SE)

(21) Appl. No.: 19/482,143

(22) PCT Filed: May 7, 2024
(86) PCT No.: PCT/SE2024/050434

§ 371 Date: Nov. 6, 2025

(30) Foreign Application Priority Data
May 9, 2023 (SE) 2350565-4

(51) Int. Cl. B03C 3/013; B01D 46/00; B01D 46/30; B03C 3/155; B03C 3/41; B03C 3/45; B03C 3/70

(52) U.S. Cl. CPC B03C 3/013; B01D 46/0028; B01D 46/0032; B01D 46/0038; B01D 46/30; B03C 3/155; B03C 3/41; B03C 3/45; B03C 3/70

(57) ABSTRACT

A hybrid filter unit includes a housing with an inlet for input of a fluid to be cleaned and an outlet for output of the fluid in a cleaned state; a wall structure arranged inside of the housing defining a mixing chamber for the fluid, wherein the wall structure defines an inner cross section area that at least partly decreases in a direction from the inlet towards the outlet; a nozzle arranged to provide an oxidizing agent to the mixing chamber so that the fluid may be subjected to the oxidizing agent and react with the oxidizing agent as it flows between the inlet and the outlet; a filtering structure arranged between an inner wall surface of the housing and an outer wall surface of the wall structure. The wall structure is at least partly adapted for through-flow of the fluid from the mixing chamber to the filtering structure.

### TECHNICAL FIELD

The invention relates to a hybrid filter unit. The term “hybrid” refers to that it is adapted for a plurality of different filtering techniques. Further, the hybrid filter unit may be used for cleaning air. More specifically, air may contain various sorts of contaminants and pollutions including organic and inorganic chemicals, Volatile Organic Compounds (VOCs), particles of different sizes, micro-organisms including various types of virus and bacteria, mould and fungi and any matter that may cause unpleasant odour or harmful effect to the environment or living beings. In different applications, there is a desire to clean the air from one or several of these contaminants and pollutions. More specifically, the hybrid filter unit may for example be used in a central ventilation system in an apartment building, office building or industrial building or as an independent plug-in air cleaner for use in a dedicated space, such as an office or private home, such as an apartment or house.

### PRIOR ART

Different types of filters are known in different filtration categories, such as physical/mechanical filtration, chemical filtration and electrical filtration. Physical/mechanical filters comprise coarse filter for coarse particles, fine filters for fine particles and ultrafine filters for ultrafine particles. Chemical filters may be categorized for ionization, oxidation, inorganic filtration and organic filtration. Electrical filters may be based on polarization (electrostatic filtration).

### SUMMARY

A first object of the invention is to provide a hybrid filter unit that is adapted for cleaning a fluid, such as air, with a high efficiency by means of at least two filtration methods and that is space-efficient while simultaneously having a pressure drop lower than the collective pressure drops of various filters in separate units.

The object is achieved by a device according to claim 1. Thus, it is achieved by hybrid filter unit comprising
a housing with an inlet for input of a fluid to be cleaned and an outlet for output of the fluid in a cleaned state,
a wall structure arranged inside of the housing defining a mixing chamber for the fluid, wherein the wall structure defines an inner cross section area that at least partly decreases in a direction from the inlet towards the outlet,
a nozzle arranged to provide an oxidizing agent to the mixing chamber so that the fluid may be subjected to the oxidizing agent and react with the oxidizing agent as it flows between the inlet and the outlet, and
a filtering structure arranged between an inner wall surface of the housing and an outer wall surface of the wall structure and wherein the filtering structure comprises volcanic Pumice, wherein the wall structure is at least partly adapted for through-flow of the fluid from the mixing chamber to the filtering structure.

According to one example, the housing comprises a wall that defines an inner space of the housing, wherein the wall structure for providing the mixing chamber is arranged in the inner space. Accordingly, the wall structure may be formed separate from the housing. According to one example, the housing wall defines generally the same cross section area along a length of the housing in a direction between the inlet and the outlet, whereas the wall structure defines a cross section area that at least partly decreases in a direction from the inlet towards the outlet.

According to one example, the wall structure has an inner cone-shaped wall that defines the mixing chamber, wherein it forms a space for mixing and for most of the chemical reactions. According to one further example, a pointy end of the inner cone-shaped wall is blocked by a transverse non-permeable wall. Due to the wall structure having an at least partly decreasing inner cross section area (being pointy) and thanks to that the pointy end of the inner cone-shaped wall is blocked, it creates a specific hydraulic flow that increases the pressure of the fluid at the blocked pointy end of the wall structure, and therefore, makes up for at least some of the filter's overall pressure drop by diverting the flow to the sides of the angled netted walls as well as making vortexes that contribute to a better mixing and longer reaction contact time. In other words, he contact time between the oxidizing agent and the incoming air is increased due to the tapering/conical shape of the inner wall, wherein fluid flow directions may change and a probability of collisions between elements in the fluid flow increases.

In an application of the hybrid filter unit, a circulating suction fan is operationally connected to the hybrid filter unit for sucking the fluid through the filter unit. In order to acquire an optimal contact time, there should be a proportionate relationship between a flow rate of the circulating suction fan (RPM) and a flow rate of the injection of the oxidizing agent.

In extreme cases, if the volumetric flow rate of the suction fan (RPM) is too high relative to the injection flow rate, there will not be enough exposure time between the oxidizing agent and the fluid inside the mixing chamber. In which case, the dosage of the oxidizing agent in the injected flow shall be high enough in the mixing chamber for it to be effective. The design of the hybrid filter unit creates conditions for increasing the probability of mixing and therefore maximizing the probability and yield of the reactions.

The factors affecting the contact time are the oxidizing agent dosage (concentration), oxidizing agent injection flow rate, fan flow rate, pollution concentration, hydraulics of the flow, hybrid filter unit housing volume, room volume (space where the air is to be cleaned), and permitted circulation frequency of the air in the room.

Further, the wording that the wall structure is at least partly adapted for through-flow of the fluid indicates that the wall structure is permeable, wherein coarse particles/matters may be collected in the hybrid filter unit. It may be achieved thanks to that a mesh size of at least one wall in the wall structure is smaller than the diameter of such coarse particles. In other words, the wall structure is arranged with a hole pattern allowing some of the particles to move through. According to one example, the wall structure comprises a wall that has a uniform hole pattern throughout its extension.

Turning now to fine particles that are not electrically charged. Most of the oxidation reactions of microorganisms release CO2 and H2O and some other products. Further, the ambient air comprises moisture and water vapor. Dust may then adsorb the moisture and gain weight so that dust particles stick together, wherein the size of the dust items increases, and they may get trapped in the hybrid filter unit, such as on the surface of the wall structure and/or downstream on the surface of the porous pumices, which are moist due to the same reasons.

Turning now to Ultra-fine particles that are not electrically charged. They may be filtered due to the combination of the oxidation process and the filtering effect of the other components including the pumice (and possibly wood fibre and coal, see further below) that takes place throughout the housing during operation in the presence of water vapor and moisture due to the two sources mentioned above (air moisture and microorganism oxidation reaction products).

Turning now to Minerals (e.g. H2S), Organic mass (VOCs), Microorganisms (bacteria, virus, mould, fungi etc.). They may be filtered due to the combination of the oxidation process and the filtering effect of the other components including the pumice (and possibly wood fibre and coal, see further below) that takes place throughout the housing.

In addition, it is known that certain chemicals, such as H2S, are sources of bad odour. Operation of the hybrid filter unit creates conditions for reduction of the bad odour in that an oxidation process caused by the oxidizing agent changes the chemical composition of the matters (e.g. leading to removing the smell) and/or physical composition (such as making them bigger since they have been oxidized and turned to a different matter). It makes the matters suitable for being filtered in the physical filtration components downstream.

Further, the hybrid filter unit creates conditions for a pressure drop minimization during the filter operation time. It may be created thanks to that a plurality of filtration processes take place in one single housing that may be compact. In contrast a total pressure drop for an arrangement of a plurality of filters for different filtration functions stacked together, one after the other, would be the sum of the filters individual pressure drops. In other words, this feature creates conditions for a pressure drop that is reduced in relation to a combination of units stacked together for the same type of operation adapted for a similar outcome of filtration and/or treatment.

According to one embodiment example, the housing is cylindrical, wherein the inlet is provided at a first end of the cylindrical housing in its axial direction and the outlet is provided at a second end of the cylindrical housing, opposite the first end, in its axial direction. According to one example, the inlet and/or the outlet is arranged so that a main axis of the inlet/outlet is in parallel with a centre axis of the cylindrical housing. According to one further example, the inlet and/or the outlet is arranged in a transverse wall defining an end of the housing in a longitudinal direction of the housing. According to one embodiment example, the housing is circular-cylindrical.

According to one embodiment example, the mixing chamber provides a space for a chemical reaction between the fluid to be cleaned and the oxidizing agent introduced in the housing by means of the nozzle. The oxidizing agent used for the reaction may in turn be generated externally of the housing.

According to a further embodiment example, the wall structure comprises a first wall and a second wall that are arranged in a spaced relationship, wherein an inner surface of the first wall defines the mixing chamber and wherein an outer surface of the second wall defines the outer wall surface of the wall structure. According to one example, each one of the first wall and the second wall is at least partly adapted for through-flow of the fluid. According to one example, the first wall and the second wall have a similar structure with regard to material and hole pattern. Further, the arrangement of two walls in a spaced relationship creates conditions for collecting particles in the spacing between the walls.

According to a further embodiment example, at least one of the first wall and the second wall has a wide end that is open to the mixing chamber and a pointy end opposite the wide end, wherein a cross section of the first wall and/or the second wall is at least partly decreasing in a direction from the wide end to the pointy end. According to one example, each one of the first wall and the second wall has a wide end that is open to the mixing chamber and a pointy end opposite the wide end.

According to a further embodiment example, the wide end of said at least one of the first wall and the second wall faces towards the inlet for allowing entry of the fluid via the wide end into the mixing chamber. According to one example, each one of the wide ends of the first wall and the second wall faces towards the inlet.

According to a further embodiment example, at least one of the first wall and the second wall is cone-shaped and wherein a base of the cone-shaped wall forms the wide end. According to one example, each one of the first wall and the second wall is cone-shaped.

According to a further embodiment example, a center axis of the cone-shaped first wall and/or a center axis of the second cone-shaped wall coincides with a center axis of the housing. According to one example, the first wall and the second wall are arranged so that the center axis of the cone-shaped first wall and the center axis of the second cone-shaped wall coincides with the center axis of the housing.

According to a further embodiment example, the wide end of said at least one of the first wall and the second wall has a cross section size and shape substantially the same as or somewhat smaller than an inner cross section shape and size of the housing for receiving substantially a complete flow of the fluid flowing between the inlet and the outlet. According to one example, the wide end of each one of the first wall and the second wall has a cross section size and shape substantially the same as or somewhat smaller than an inner cross section shape and size of the housing.

In other words, the first wall and the second wall are arranged so that a gap is formed between an outer surface of the first wall and an inner surface of the second wall. According to one example, the second wall defines a more acute tip than the first wall, wherein the gap between the walls increases in a direction from the wide end towards the pointy end. It creates further space for collection of matter between the walls.

According to one example, the inner cone-shaped wall defines the mixing chamber that forms a space for mixing and for most of the chemical reactions. The first wall itself may be designed so that it does not adsorb the minerals and microorganisms and other substances in the fluid to be treated. However, it contains them and lets them react with the oxidizing agent. The substances that are not reacted may get trapped in the housing thanks to the other filtering arrangements.

As mentioned above, the filtering structure comprises a plurality of items of volcanic Pumice that are at least partly rounded and have porosities. It provides a substantial surface area leading to an increased contact area and increased contact time during operation and therefore increased absorption of the substances. In addition, the volcanic pumice can act like a catalyst and facilitate the chemical reactions as well as increasing the contact time and reaction surface area due to its inherent chemical properties and the minerals that it contains in its porosities. Further, the filtering structure may comprise dried wood fibre and/or activated carbon that is provided between the items of volcanic Pumice. The pumice porosities may contain enough moisture to facilitate the chemical reactions during operation. The humidity of the ambient air (fluid flow) and/or the produced water due to chemical reactions are responsible for the availability of the mentioned moisture. If the oxidizing agent is not present in the filtering section downstream, some microorganism colonies might get the possibility to grow inside the wood fibre or carbon active or pumice section, especially, if the pumice is not volcanic, the growth chance is higher. Therefore, in this design it is considered that the oxidizing agent has the ability to be present also in the filtering media downstream to avoid the growth of microorganisms as well as preventing the spread of any free radical atom/ion to the room whose air is being cleaned.

The minerals and microorganisms and all the other mentioned substances that do not have enough contact time to react with the oxidizing agent flow through the wall structure to the pumice, wherein the pumice with the porosities and high surface area provides enough contact time for the chemical reactions to complete the oxidation of the remaining substances and increases the probability of the substances being physically trapped in the filter.

Thanks to the roundness of the pumice items, the pressure drop is minimized, wherein it creates conditions for a lower power consumption and/or more cost-efficient unit in that a suction fan arranged to suck the fluid through the pumice may be of smaller size. In other words, it is more cost-efficient in that a reduced amount of energy is required during operation compared to a combination of units stacked together. This is one determining factor for choosing a type of filtering technologies and filtering products.

The dried wood fibre and activated carbon may be adapted for calibrating the pressure drop. In other words, by altering the amount of the dried wood fibre and activated carbon for different applications, the pressure drop can be regulated/calibrated, according to the application of the unit and the target quality of the cleaned air, e.g. home/hospital/clean room. According to one example, a minority of active carbon and wood fibres may be used in between the pumices to fill in some of the voids between the pumices. These are optional items to boost the absorption efficiency as well as adjusting the pressure drop.

The active size of the carbon that may be used here is preferably in a range of from 0.2 mm to 1.8 mm. The wood fibre is a string with various lengths and the cross section of the string looks like almost a rectangle with the dimensions of 0.1*2 mm.

In addition, volcanic pumice has some mineral properties (presence of mineral matters such as Sulphur and manganese (Mn) and magnesium (Mg). It prevents the growth of some of microorganisms (for example algae). The individual pumice items have a size with a main extension/diameter between 20 to 50 mm and preferably around 50 mm.

Further, the hybrid filter unit creates conditions for noise minimization thanks to the arrangement of the pumice and the pointy shape of the cone-shaped walls and therefore, change of direction of sound waves. Further, the filtering structure that contains the volcanic pumice (and possibly the and the wood fibre and activated carbon) may adsorb ultrafine particles.

According to a further embodiment example, the hybrid filter unit comprises an arrangement for attracting charged particles in the fluid flow, wherein the arrangement is arranged inside of the housing, wherein the arrangement for attracting charged particles comprises an inner electrically conducting element and an outer electrically conducting element arranged in a spaced relationship, and wherein the inner electrically conducting element and the outer electrically conducting element are adapted for being connected to a power source for being oppositely charged so that electrically charged particles in the fluid are attracted to one of the electrically conducting elements, wherein the power source is adapted to supply a low voltage and wherein it is connected so that the inner electrically conducting element is positively charged. Some particles in the fluid flow may become charged due to the chemical reaction taking place in the housing when the fluid is subjected to the oxidizing agent. Other particles in the fluid flow may already be charged as the fluid enters the housing. The voltage being low means that it only makes the cones electrostatically charged while not creating conditions for any electrical discharge such as glow or corona etc. The avoidance of discharge is both for safety and presence of carbon and other flammable physical structures present in common filters.

Accordingly, the inner electrically conducting element (that may be adapted to form an inner cone) is positively charged and the outer electrically conducting element (that may be adapted to form an outer cone) is negatively charged during operation. According to one example, the inner electrically conducting element is exclusively connected to the positive pole of a power source adapted for 24 volts (+24 v) and the outer electrically conducting element is connected to the negative pole of the 24 volts power source (−24 v).

When the space between the two (cone-shaped) electrically conducting elements is filled up with particles/matter, it needs to be emptied. Thanks to the fact that the two electrically conducting elements are positively and negatively charged, respectively, it may be used as a sensor that may alarm when it needs to be emptied. More specifically, a control circuit may be connected to the two electrically conducting elements. If short circuit starts to happen, the amperage starts increasing and when it reaches a predetermined threshold indicating a risk of a fire, the control circuit discharges the positively charged cone from the positive pole and then the negatively charged cone from the negative pole, wherein the voltage difference between the two cones becomes zero. Then an error is communicated to clean the space between the two electrically conducting elements by an operator of maintenance.

According to a further embodiment example, the arrangement for attracting charged particles is arranged downstream of the nozzle in a direction of fluid flow between the inlet and the outlet. Accordingly, the hybrid filter unit creates conditions for a sequence of first electrically charging the particles and then attracting the particles as they flow through the housing. In other words, the hybrid filter unit creates conditions for a polarization process during operation, wherein the particles that have acquired electrical charges due to reaction with the oxidizing agent in the mixing chamber are adsorbed to the electrically conducting elements of the opposite charge. Since some of the particles are negatively charged, they are attracted to the positively charged electrically conducting element and the other ones that are positively charged are attracted to the negatively charged electrically conducting element.

According to a further embodiment example, at least one of the inner electrically conducting element and the outer electrically conducting element forms one of said first wall and second wall of the wall structure defining the mixing chamber. Accordingly, the arrangement for attracting charged particles may be integrated in the wall structure. It creates conditions for a space-efficient unit. According to one example, the inner electrically conducting element forms said first wall and the outer electrically conducting element forms the second wall of the wall structure defining the mixing chamber.

According to a further embodiment example, the inner electrically conducting element is at least partly arranged inside of the outer electrically conducting element, wherein the inner electrically conducting element and the outer electrically conducting element are arranged so that particles may be collected in a spacing between the inner electrically conducting element and the outer electrically conducting element.

According to a further embodiment example, the outer electrically conducting element is arranged downstream of the inner electrically conducting element in a fluid flow direction. Accordingly, the space between the first wall and the second wall is empty at an initial state of operation so that it can trap the dust and some particles during operation. It may be noted that not only the charged particles but also the minerals and organic substances including the micro-organisms, TVOC and products and by-products (liquids such as water and acids) of the chemical reactions of the pollutants in the incoming fluid and the oxidizing agent may be trapped and/or adsorbed in the space.

Accordingly, the substances that are electrically charged, fail to reach the pumice since they are adsorbed and trapped by the polarization process of the two cone-shaped walls. On the other hand, the particles that are not charged enough to be adsorbed by the two cone-shaped walls, go through both of the two cone-shaped walls and reach the filtering structure and get exposed to the pumice and possibly the active carbon and wood fibres and the existing moisture.

According to a further embodiment example, the hybrid filter unit comprises an element providing electrical insulation between the inner electrically conducting element and the outer electrically conducting element. According to one example, the electrically insulating element is provided adjacent the wide ends of the electrically conducting elements. More specifically, the electrically conducting elements may be removably attached to the electrically insulating element for ease of disassembly.

According to a further embodiment example, the housing comprises a first open end, wherein the hybrid filter unit comprises a first cap that defines an opening and is adapted to be connected to the first open end of the housing in a way that the cap opening forms the inlet of the housing, wherein at least one of the electrically conducting elements is attached to the cap. According to one example, the end cap is adapted to hold both (cone-shaped) electrically conducting elements, wherein the hybrid filter unit is easily opened, the two (cone-shaped) electrically conducting elements separated and the space emptied for maintenance.

According to a further embodiment example, at least one of the first electrically conducting element and the second electrically conducting element is releasably attached to the first cap for allowing access to a spacing between the first electrically conducting element and the second electrically conducting element for cleaning.

According to a further embodiment example, the first cap forms the electrically insulating element between the first electrically conducting element and the second electrically conducting element.

According to a further embodiment example, the housing comprises a second open end, wherein the hybrid filter unit comprises a second cap that defines an opening and is adapted to be connected to the second open end of the housing in a way that the cap opening forms the outlet of the housing. Further, the second cap may be arranged to contact the pumice and pack the pumice and possibly other contents of the filter structure downstream of the cone-shaped wall structure.

According to a further embodiment example, the nozzle is arranged in a wall of the housing so that its inner end is located inside of the housing and a body of the nozzle extends transversally relative to a center axis of the housing between the inlet and the outlet. Further, the nozzle inner end may be arranged inside of the mixing chamber defined by the wall structure.

According to a further embodiment example, the hybrid filter unit comprises a means for drainage of by-products resulting from the reaction of the fluid with the oxidizing agent. The by-products may be water and other liquids, such as carbonic acid but depending on the pollutants in the incoming air, the liquid can contain other chemicals as well that are the by-products of the reaction between the oxidizing agent and the pollutants. A majority of possible by-products are acidic but usually weak acids. In addition, there could be also solid by-products such as various types of mineral salts that can also be dissolved in the liquid by-products and be drained.

Since the shape of the filter is cylindrical, the liquid is more drawn to the bottom of it (lowest point in vertical direction) and therefore, the nozzle shall be at the bottom of the filter so it can maximize the emptying.

According to a further aspect of the invention, it regards a fluid cleaning arrangement comprising a device for generating an oxidizing agent, and a hybrid filter unit according to any preceding embodiment and example, wherein the oxidizing agent generating device is arranged separate from the hybrid filter unit and an outlet of the oxidizing agent generating device is operatively connected to the nozzle for providing the oxidizing agent to the hybrid filter unit. According to one example, the oxidizing agent generating device is a Branilium™ reactor.

According to one embodiment example, the oxidizing agent generating device is adapted for generating the oxidizing agent by subjecting a further fluid for an electric discharge/Plasma so that it is ionized.

According to a further embodiment example, the oxidizing agent generating device is adapted for generating the oxidizing agent by subjecting air for an electric discharge/Plasma independent to ground/earth electrode or pole.

According to a further embodiment example, the fluid cleaning arrangement comprises an external air source arranged separate from the oxidizing agent generating device, wherein the external air source is arranged to receive air from outdoors, wherein the external air source is operationally connected to an inlet of the oxidizing agent generating device for providing the received outdoors air to the oxidizing agent generating device for reducing risks associated to using polluted indoors air.

The air for the oxidizing agent generating device may be ambient air collected from outside of the space/building that contains the air to be cleaned. Accordingly, the air for the oxidizing agent generating device and the air to be cleaned by means of the hybrid filter unit may be collected from different sources.

In hybrid air purification systems, the ionization process involves the emission of electrons and/or positrons (electrical discharges) from an electrode under the influence of a high alternating current (AC) voltage. This high-voltage application is critical as it provides the necessary energy to overcome the ionization potential of the molecules, primarily oxygen in the air around the electrodes. When the AC voltage is applied, it creates a strong electric field within the chamber or around the electrodes. This field accelerates free electrons to energies sufficient to ionize oxygen and other gases present, leading to the formation of ions and various reactive oxidant species.

On the other hand, the hybrid filter unit can also utilize external sources of oxidants, such as chemical capsules containing chlorine (Cl2), titanium dioxide (TiO2), or formalin, which are injected directly into the chamber without relying on ionization. These substances act as potent oxidizing agents that chemically alter and neutralize airborne contaminants. This method does not depend on the generation of ions through electrical discharge, offering an alternative mechanism for air purification that can be effective in different contexts.

The generated ions and reactive species are integral to the air purification process, as they can effectively neutralize pollutants, pathogens, and odors. It's important to note that the term “ionizer/anion generators” in commercial air purifiers can be misleading. While many devices are marketed as ionizers, implying a benign generation of ions, some may produce significant amounts of ozone as a byproduct, which is a potent oxidant but hazardous to health in sufficient concentrations. The effectiveness and safety of these systems depend on their design and the specific nature of the electrical discharges they employ. Properly engineered systems ensure that ion generation enhances air quality without elevating ozone to harmful levels.

It's pertinent to address the limitations of certain air purifiers that employ direct current (DC) and low voltage transformers. By physical law, these systems typically do not deliver sufficient energy for effective ionization. Ionization requires that the electrons gain enough kinetic energy to detach from their atoms and ionize surrounding gas molecules. DC systems and low-voltage setups often fall short of this energy threshold needed to initiate and sustain the ionization process.

If the claim were valid that simple DC and low-voltage systems could adequately ionize air, then common household devices like mobile chargers and electrical plugs, which also operate at these lower voltages (around 230V in many regions), would inherently ionize the air. This would imply that all indoor spaces with such devices would be automatically purified. However, this is not observed in practical scenarios. The lack of widespread ionization in everyday environments with these devices underscores the necessity for specific high-voltage AC systems in air purifiers designed to achieve effective ionization. These high-voltage systems create the conditions necessary for electron acceleration and subsequent air ionization, which are not replicable by standard DC-powered devices or low-voltage electrical systems.

According to a further embodiment example, the fluid cleaning arrangement comprises a mounting frame, wherein the ionization device and the hybrid filter unit are fixedly connected to the mounting frame in a side-by-side relationship. According to one example, attachment portions in opposite ends of the ionization device and the hybrid filter unit are adapted to be attached to corresponding attachment portions in opposite spaced walls of the mounting frame.

The arrangement comprising the device for generating an oxidizing agent being external in relation to the hybrid filter unit creates conditions for avoiding accumulation of oil and dust in the hybrid filter unit and avoiding of the filter structures and oxidizing agent nozzle becoming greasy. It creates conditions for a higher efficiency (yield) of the oxidizing agent. It further creates conditions for the oxidizing agent being generated with higher efficiency and be independent of the pollutions in the fluid to be cleaned.

According to one example, the oxidizing agent contains radicals and substances with free electrons and positrons. Such particles (electrons and positrons and possibly quarks and/or muons) are accordingly free and available. The electrons and positrons possess negative and positive electrical charge, respectively, and they can attach/induct to the dust and other fine particles in the air to make them electrically charged. According to one example, such an oxidizing agent may be created in an ionization process taking place separate from the hybrid filter unit.

Physical particles in the air, such as wool, dust, human or pet hair, acquire electrical charges due to reaction with the oxidizing agent. Some of the dust particles are already electrically charged (due to friction) in the air but mixing with the oxidizing agent may reinforce the charge.

Turning now to the arrangement for attracting charged particles in the fluid flow. The opposite charge of the first and second electrically conducting elements creates conditions for polarization utilized to adsorb dust and fine particles.

To summarize, the hybrid filter unit creates conditions for the following steps during operation:
1. some of the dust particles can get electrically charged and adsorbed to the surface of the electrically charged elements through an electrostatic mechanism,
2. some other particles that are not electrically charged, are adsorbed to the wall structure due to gaining weight,
3. some other particles who have not been adsorbed by the first two steps above, after a few rounds of circulation of the air inside a room by a suction fan, at some point they will get stuck in the wall structure anyway.

Further advantages and advantageous features of the invention are disclosed in the following description and in the dependent claims.

### BRIEF DESCRIPTION OF THE DRAWINGS

With reference to the appended drawings, below follows a more detailed description of embodiments of the invention cited as examples.

In the drawings:

FIG. 1 is a schematic perspective view of a hybrid filter unit according to a first embodiment,

FIG. 2 is an exploded view of the hybrid filter unit according to FIG. 1,

FIG. 3 is a cross section view of the hybrid filter unit according to FIG. 1 with added components,

FIG. 4 is a schematic cross section view of the hybrid filter unit according to FIG. 1, wherein a hydraulic fluid flow is indicated.

FIG. 5 is a schematic perspective view of a fluid cleaning arrangement according to a first embodiment, that comprises the hybrid filter unit according to FIG. 1 and a device for generating an oxidizing agent,

FIG. 6 is a partly cut and exploded perspective view of the device for generating an oxidizing agent in FIG. 5,

FIG. 7 is a perspective view of a mounting frame, wherein the hybrid filter unit and the device for generating an oxidizing agent according to FIG. 5 are indicated in a mounted state,

FIG. 8 is a schematic perspective view of a hybrid filter unit according to a second embodiment,

FIG. 9 is a perspective view of a mounting frame, wherein two hybrid filter units, each with an associated device for generating an oxidizing agent, are indicated in a mounted state,

FIG. 10 is a cross section view of an ionization unit comprised in the device for generating an oxidizing agent in FIG. 6, and

FIG. 11 is an alternative embodiment of an ionization unit.

### DETAILED DESCRIPTION OF EXAMPLE EMBODIMENTS

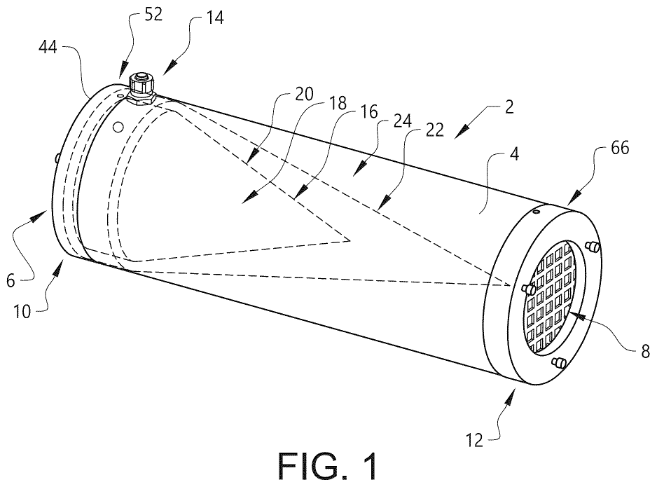

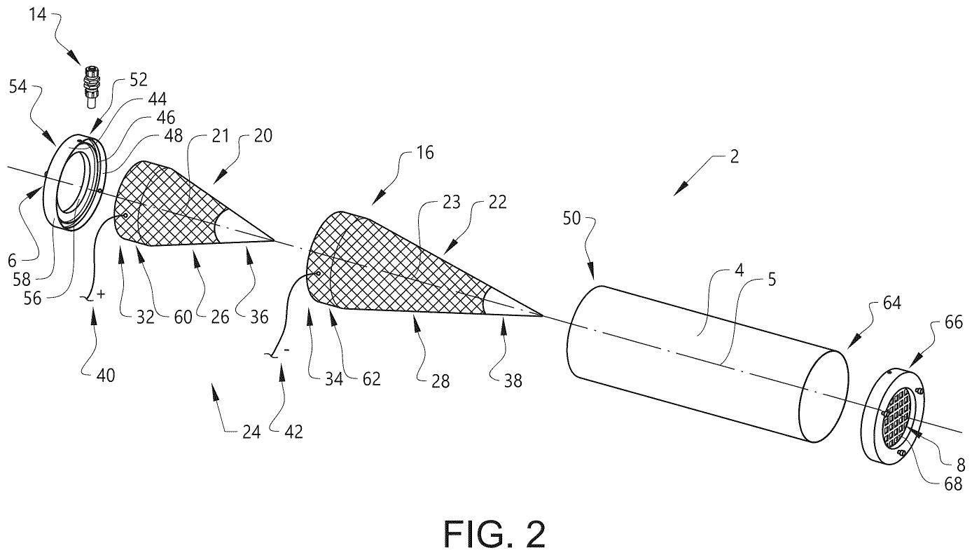

FIG. 1 is a schematic perspective view of a hybrid filter unit 2 according to a first embodiment. FIG. 2 is an exploded view of the hybrid filter unit 2 according to FIG. 1. The hybrid filter unit 2 comprises a housing 4 with an inlet 6 for input of a fluid to be cleaned and an outlet 8 for output of the fluid in a cleaned state.

The housing 4 is cylindrical, wherein the inlet 6 is provided at a first end 10 of the cylindrical housing 4 in its axial direction and the outlet 8 is provided at a second end 12 of the cylindrical housing 4, opposite the first end 10, in its axial direction. More specifically, the housing 4 is circular-cylindrical. Each one of the inlet 6 and the outlet 8 has an axis in parallel with the axial direction of the cylindrical housing. More specifically, each one of the inlet 6 and the outlet 8 is arranged in an end wall defining an interior of the housing 4 in the axial direction.

The hybrid filter unit 2 comprises a nozzle 14 arranged to provide an oxidizing agent to a space inside of the housing 4 so that the fluid may be subjected to the oxidizing agent and react with the oxidizing agent as it flows between the inlet 6 and the outlet 8. The oxidizing agent may be provided from various types of sources such as stored in a capsule or generated adjacent and possibly simultaneously as the operation of the hybrid filter by the means of a reactor or oxidant generator, etc.

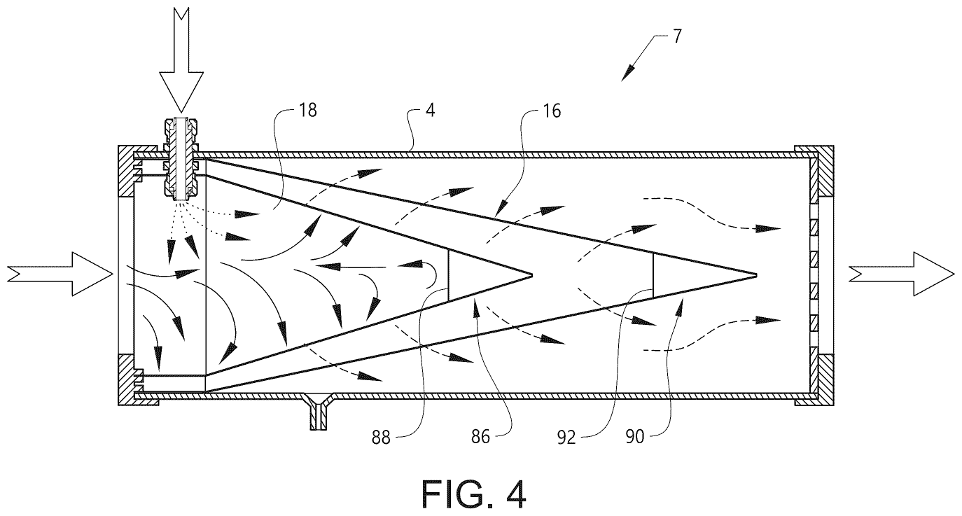

The hybrid filter unit 2 further comprises a wall structure 16 arranged inside of the housing 4 defining a mixing chamber 18 for the fluid. The wall structure 16 has a through-flow area that at least partly decreases in a direction from the inlet 6 towards the outlet 8. More specifically, the wall structure 16 comprises a first wall 20 that defines the mixing chamber 18 in a transverse direction of the housing 4. More specifically, the first wall 20 defines the mixing chamber 18 in a radial direction of the cylindrical housing 4. More specifically, the first wall 20 has a through-flow area that decreases in a direction from the inlet 6 towards the outlet 8. More specifically, the first wall 20 has a conical shape. Such a tapering shape of the inner wall 20 has the effect that the fluid flow and the particles in the flow may change directions, see also FIG. 4, wherein it provides for a uniform mixing.

The wall structure 16 further comprises a second wall 22 that is arranged in the vicinity of the first wall 20 and spaced from the first wall 20 in an axial direction of the housing 4 so that a gap is formed between the first wall 20 and the second wall 22. More specifically, also the second wall 22 has a conical shape. A wide end of each one of the first conical wall 20 and the second conical wall faces the inlet 6.

Each one of the first and second walls 20, 22 of the wall structure 16 is at least partly adapted for through-flow of the fluid, i.e. permeable. More specifically, each one of the first and second walls 20, 22 of the wall structure 16 comprises a hole pattern in the form of a net or grid allowing the fluid to pass through the respective wall. More specifically, each one of the first and second walls 20, 22 of the wall structure 16 comprises the hole pattern throughout its extension.

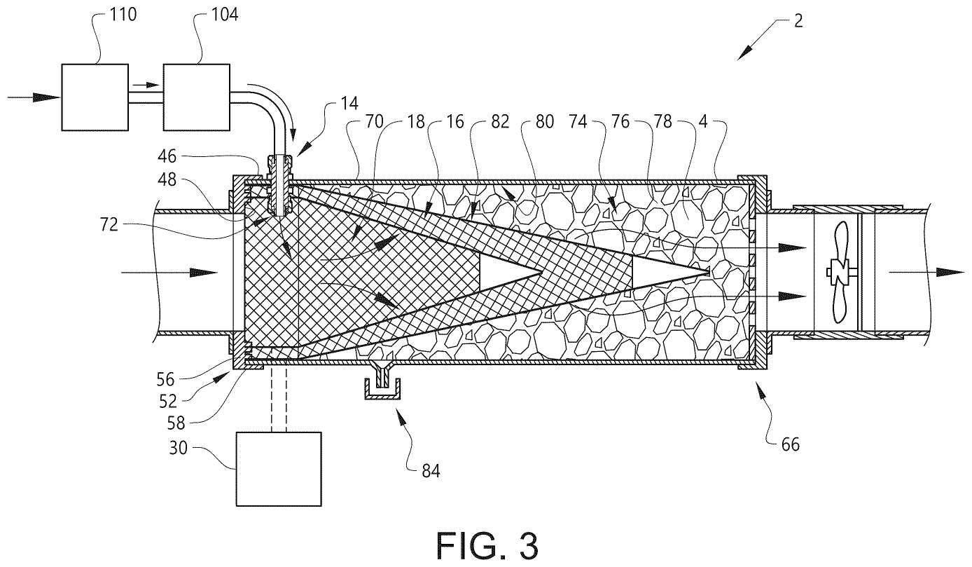

The hybrid filter unit 2 further comprises an arrangement 24 for attracting charged particles in the fluid flow. The arrangement 24 is arranged inside of the housing 4, wherein the arrangement for attracting charged particles comprises a first electrically conducting element 26 and a second electrically conducting element 28 arranged in a spaced relationship. The first electrically conducting element 26 and the second electrically conducting element 28 are adapted for being connected to a power source 30, see FIG. 3, for being oppositely charged, see plus sign 40 and minus sign 42 in FIG. 2, so that electrically charged particles in the fluid are attracted to one of the electrically conducting elements 26, 28.

The arrangement 24 for attracting charged particles is arranged downstream of the nozzle 14 in a direction of fluid flow between the inlet 6 and the outlet 8. More specifically, each one of the first electrically conducting element 26 and the second electrically conducting element 28 forms one of the first wall 20 and the second wall 22 of the wall structure 16 defining the mixing chamber 18.

Accordingly, each one of the first electrically conducting element 26 and the second electrically conducting element 28 has a wide end 32, 34 that is open to the mixing chamber 18 and a pointy end 36, 38 opposite the wide end, wherein a cross section of the electrically conducting element is decreasing in a direction from the wide end 32, 34 to the pointy end 36, 38. Further, the wide end 32, 34 of each one of the first electrically conducting element 26 and the second electrically conducting element 28 faces towards the inlet for allowing entry of the fluid via the wide end into the mixing chamber 18. In other words, each one of the first electrically conducting element 26 and the second electrically conducting element 28 is cone-shaped, wherein a base of the cone-shaped electrically conducting element 26, 28 forms the wide end.

Further, a center axis 21 of the cone-shaped first electrically conducting element 26 and a center axis 23 of the second electrically conducting element 28 coincides with a center axis 5 of the housing 4.

Further, the wide end 32, 34 of said at least one of the first electrically conducting element 26 and the second electrically conducting element 28 has a cross section size and shape substantially the same as or somewhat smaller than an inner cross section shape and size of the housing 4 for receiving substantially a complete flow of the fluid flowing between the inlet 6 and the outlet 8.

More specifically, the first electrically conducting element 26 is at least partly arranged inside of the second electrically conducting element 28, wherein the first electrically conducting element 26 and the second electrically conducting element 28 are arranged so that dust and/or fine particles may be collected in a spacing between the first electrically conducting element 26 and the second electrically conducting element 28. More specifically, the second electrically conducting element 28 is arranged downstream of the first electrically conducting element 26 in a center axis direction of the housing between the inlet 6 and the outlet 8.

The hybrid filter unit 2 further comprises an element 44 providing electrical insulation between the first electrically conducting element 26 and the second electrically conducting element 28 at the wide end 32, 34 of the elements 26, 28. The electrical insulation element 44 has an annular shape and comprises two radially spaced receptacles 46, 48 for receiving the wide ends 32, 34 of the first electrically conducting element 26 and the second electrically conducting element 28. More specifically, each one of the receptacles 46, 48 is formed by a circular slot. Each one of the first electrically conducting element 26 and the second electrically conducting element 28 comprises a cylindrical end portion 60, 62 of constant diameter for receipt in the circular slot 46, 48.

The housing 4 comprises a first open end 50, wherein the hybrid filter unit comprises a first cap 52 that defines an opening 54 and is adapted to be connected to the first open end 50 of the housing 4 in a way that the cap opening 54 forms the inlet 8 of the housing. More specifically, each one of the electrically conducting elements 26, 28 is attached to the first cap 52. According to the first embodiment, the first cap 52 forms the electrical insulation element 44.

Further, the first cap 52 is releasably attached to the housing 4. The first cap 52 comprises an annular wall 56 with an extension perpendicular to the center axis 5 of the housing 4 and a cylindrical portion 58 with an axis in parallel with the center axis 5 of the housing 4. The annular wall 56 and the cylindrical portion 58 are formed in a one-piece unit. The cylindrical portion 58 has a somewhat larger inner diameter than an outer diameter of the housing 4 for achieving a tight fit. Each one of the first electrically conducting element 26 and the second electrically conducting element 28 is releasably attached to the first cap 52 for allowing access to a spacing between the first electrically conducting element 26 and the second electrically conducting element 28 for cleaning.

The housing 4 further comprises a second open end 64, wherein the hybrid filter unit 2 comprises a second cap 66 that defines an opening 68 and is adapted to be connected to the second open end 64 of the housing 4 in a way that the cap opening 68 forms the outlet 8 of the housing.

Turning now to FIG. 3 that shows a cross section view of the hybrid filter unit 2. The nozzle 14 is arranged in a wall 70 of the housing 4 so that its inner end 72 is located inside of the housing 4 and a body of the nozzle extends transversally relative to the center axis 5 of the housing 4. More specifically, the nozzle inner end 72 is arranged inside of the mixing chamber 18 defined by the wall structure 16.

The hybrid filter unit 2 further comprises a filtering structure 74 arranged between an inner wall surface 80 of the housing 4 and an outer wall surface 82 of the wall structure 16. More specifically, the filtering structure 74 is arranged downstream of the arrangement 24 for attracting charged particles in the direction of fluid flow between the inlet 6 and the outlet 8. The filtering structure 74 comprises a plurality of filtering elements 78 that are at least partly rounded and have porosities. More specifically, the filtering structure 74 comprises volcanic Pumice. More specifically, the filtering structure 74 comprises dried wood fibre and/or activated carbon 76 that is provided between items of volcanic Pumice.

The hybrid filter unit 2 comprises a means 84 for drainage of by-products resulting from the reaction of the fluid with the oxidizing agent. The drainage means 84 defines an opening through the wall 70 of the housing 4 in a bottom of the housing 4.

The arrows in FIG. 3 indicate the fluid flow and the flow of oxidizing agent injected in the hybrid filter unit 2.

FIG. 4 is a schematic view of the hybrid filter unit 2 according to FIG. 1, wherein the hydraulics of a fluid flow is indicated in more detail. It may be noted that an inner pointy end 86 of the inner conical wall 20 is closed by an impermeable wall 88 extending in a direction transverse to the center axis 21 of the inner conical wall 20. In this way, a portion of the incoming fluid flow will turn around and provide for increased mixing in the mixing chamber 18. In a similar way, an inner pointy end 90 of the outer conical wall 22 is closed by an impermeable wall 92 extending in a direction transverse to the center axis 23 of the outer conical wall 22. It creates further conditions for an increased mixing in the mixing chamber 18. Thanks to that the ends of the conical walls 20, 22 are blocked, it increases the pressure of the fluid and therefore makes up for at least some of the pressure drop as well as making vortexes that contribute to a better mixing and longer reaction contact time.

As an example, the air to be treated may be provided in a flow of about 1000 L/min while the oxidizing agent may be provided in a flow of about 50 L/min. Since the magnitude of the air flow is bigger than the oxidizing agent flow, it is the dominant flow and can bend the oxidizing agent flow in the flow direction towards the tips/pointy ends of the conical walls 20,22. This interaction between the air flow and the oxidizing agent flow creates mixing between them and allows for their respective substances to chemically or physically react with each other. As examples of physical reactions, the following can be mentioned: Electrical charge exchange or adsorption on surface or even generating electrical charge due to friction with one another. Example of chemical reaction could be oxidation reaction of the substances of the air flow due to presence of oxidizing agent in the oxidizing agent flow.

For example, if the oxidant concentration is 20 mg/L (ppm), the exposure dosage would be 50×20=1000 mg/min or 1 g/min or 60 g/h. For disinfection of the air of an office, this might be enough but for more polluted applications, the dosage shall be adjusted through circulation rate (reducing the fan speed from 1000 L/min or increasing the oxidant concentration from 20 mg/L to a higher level by improving the efficiency of the oxidant agent generating process.

This ability of being flexible enough to be able to adjust these parameters in a space efficient way depending on the various flow rates as well as pollution levels, is an advantage in having a separate (external) source of oxidizing agent. Accordingly, it is possible to adjust the injection flow and all the mentioned parameters such as the oxidant concentration and the fan speed accordingly.

In addition, a suction fan (not shown) may be connected to the outlet of the housing for sucking the air flow through the pumice and wooden fibres inside the housing.

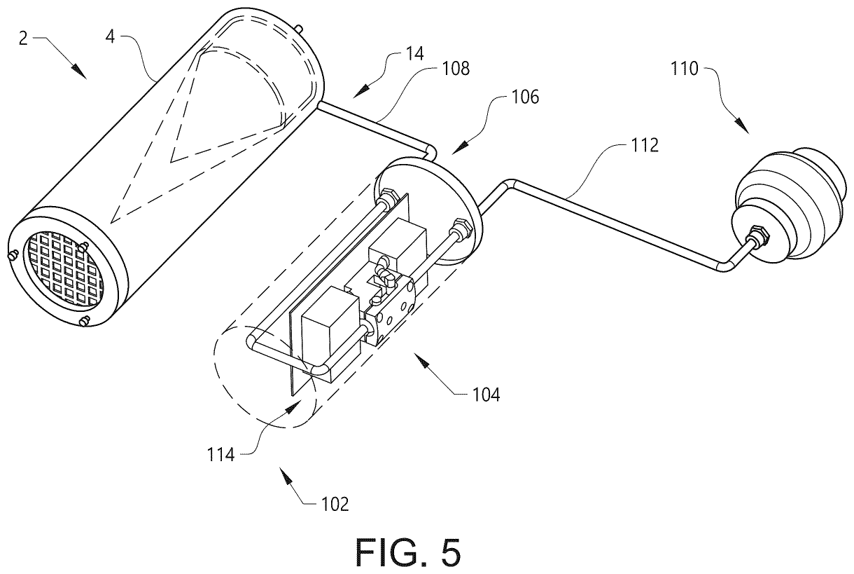

FIG. 5 is a schematic perspective view of a fluid cleaning arrangement 102 according to a first embodiment. The fluid cleaning arrangement 102 comprises the hybrid filter unit 2 according to FIG. 1 and a device for generating an oxidizing agent 104. The device for generating an oxidizing agent 104 is adapted for ionization of a further fluid for achieving an output flow of an oxidizing agent. The further fluid is here in the form of air. The device 104 is arranged separate from the hybrid filter unit 2 and an outlet 106 of the device 104 is operatively connected to the nozzle 14 via a tube or hose 108 for providing the oxidizing agent to the hybrid filter unit 2.

The fluid cleaning arrangement 102 further comprises an air compressor 110 operatively connected to the device 104 for providing the device 104 with compressed air.

The device for generating an oxidizing agent 104 is arranged in a casing that has a generally circular cylindrical outer shape. The device for generating an oxidizing agent 104 comprises a generally flat rectangular wall 118 and wall 120 that is generally half-circular in cross section, which is connected to the flat rectangular wall 118 in a way defining an internal space between the walls 118, 120. An ionization unit 114 is arranged in the internal space between the walls 118, 120.

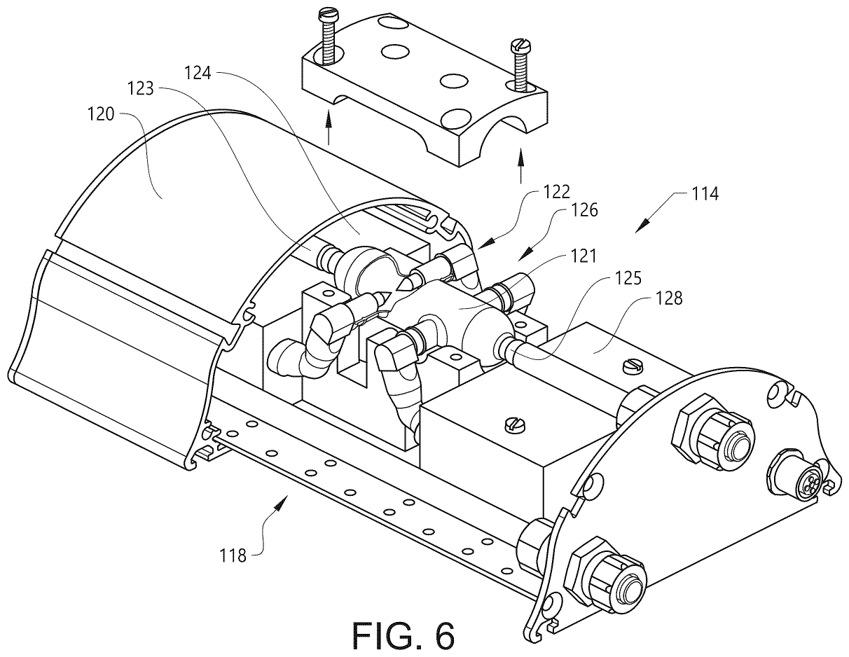

FIG. 6 is a partly cut perspective view of the ionization unit 114 in FIG. 5. The ionization unit 114 is adapted for subjecting the compressed air to an electric discharge for achieving the output flow. The ionization unit 114 comprises an elongated container 121. The container 121 has a rounded cross section shape and more specifically a circular cross section shape. Further, the cross section of the container 4 is constant along a significant portion of the length of the container. Further, ends of the container 121 in the longitudinal direction have a rounded and more specifically half-spherical shape. A wall of the container 121 defines an inner chamber. The container wall is formed in glass. Further, an inlet 123 is provided at a first end of the container 121 in its longitudinal direction and an outlet 125 is provided at a second end of the container in its longitudinal direction for conveying a gas flow from the inlet 123 to the outlet 125.

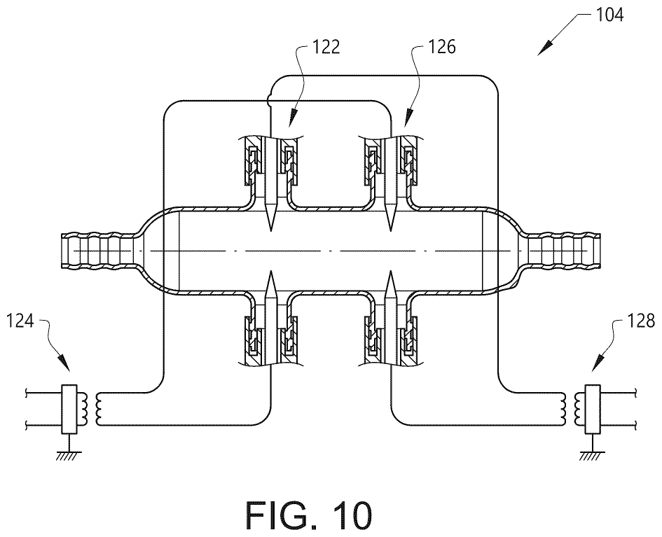

FIG. 10 is a cross section view of the ionization unit 114 in FIG. 6.

The ionization unit 114 further comprises a first pair of electrodes 122 arranged in the container opposite each other and at a distance from each other. Each one of the electrodes in the first pair 122 has an elongated shape with a circular cross section and a pointy end. The electrodes are arranged so that the pointy ends face each other.

According to physics law, when charging an element, the charged parts accumulate in any sharp edges of the element. Accordingly, charged parts will be highly accumulated in the sharp edge of the electrode. In other words, charged parts will have a very high density in the sharp edge, wherein an electric field will be strong in a region of the sharp edge. Further, a highly charged electrode (positive or negative) will have a very high potential in relation to the environment (adjacent the electrode). The potential difference between the electrode and its adjacent environment/surroundings will result in ionization of the matter in the environment in the vicinity of the respective electrode leading to exchange of electrons/positrons in cycles from higher potential area to lower potential area and vice versa and different types of electric discharge from the electrode may take place. This phenomenon may be similar to a Tesla coil.

Accordingly, the design of the electrodes with sharp tips especially with an acute angle from 20 to 35 degrees (preferably 22 degrees to create a greater number of electric discharges as well as increasing the life span of the electrodes) creates good conditions for creating electric discharges from the surface of the tip having an inclination relative to the longitudinal direction of the elongate electrode.

The ionization unit 114 further comprises a power supply 124, 128 adapted for charging each one of the electrodes in the first pair 122 of electrodes so that they have the same charge simultaneously. In this way, such a potential difference between each one of the electrodes and an environment of the respective electrode may be created that electric discharges take place simultaneously from each one of the electrodes. Further, the fluid is conveyed in a gaseous state inside the container past the first pair 122 of electrodes in the environment of the respective electrode during said charging for ionization of the fluid.

More specifically, the power supply 124, 128 comprises two transformers 124, 128, which are adapted to provide an alternating current of a certain frequency to the electrodes. Accordingly, the power supply 124, 128 is adapted to supply such a voltage to the first pair 122 of electrodes that both electrodes are positively charged at the same time and therefore emit electrons. Further, the container is adapted for conveying the gas in a flow past the first pair 122 of electrodes, wherein the gas flow may be regarded as a negatively charged region between the electrodes for interaction with the emitted electrons from the electrodes so that a first electric discharge structure may be created. More specifically, a plurality of electric discharges project from each one of the electrodes for ionization of the gas.

More specifically, each transformer 124, 128 comprises a primary winding and a secondary winding. Each transformer turns the voltage from an input of 12 to 220 volts with a frequency of 50 to 60 Hz to 2*7.5 kV for each pole (associated to one of the electrodes) with a frequency of about 20 kHz by changing the electric charge of the electrodes (AC current). Accordingly, each transformer 124, 128 comprises a frequency converter where one of the ground wire functions is to reduce noise.

Each one of the transformers 124, 128 is adapted for supplying an output voltage at a magnitude of around 7.5 kV via each one of its output terminals. Further, each one of the transformers 124, 128 is adapted for supplying the output voltage in a frequency about 20 kHz, wherein the polarity of the electrodes connected to the two output terminals/poles of one transformer will change very fast (every 0.00005 second).

More specifically, each one of the electrodes is arranged in an opening through the container wall. More specifically, the container comprises a pipe-shaped portion extending in a transverse direction relative to the longitudinal direction of the container. More specifically, the pipe-shaped portion extends perpendicularly relative to the longitudinal direction of the container. The pipe-shaped portions define the openings. More specifically, the pipe-shaped portions are formed in one-piece with the container. More specifically, the electrodes are arranged in the pipe-shaped portions in a gas tight manner for avoiding leakage.

The ionization unit 114 further comprises a second pair 126 of electrodes arranged in the container in a similar way as has been described above with regard to the first pair 122 of electrodes. The second pair 126 of electrodes are arranged at enough distance from the first pair 122 of electrodes in the longitudinal direction of the container. Each one of the first pair 122 of electrodes and the second pair 126 of electrodes are arranged at the portion of the container having a constant cross section with a distance between the adjacent electrode pairs of about 30 mm. The power supply 124, 128 is adapted for charging each one of the electrodes in the second pair 126 of electrodes so that they have the same charge at the same time. In this way, such a potential difference between each one of the electrodes and an environment of the respective electrode may be created that electric discharges take place from each one of the electrodes separately. Accordingly, the power supply 124, 128 is adapted to supply such a voltage also to the second pair 126 of electrodes that both electrodes are positively charged at the same time and therefore emit/exchange electrons/positrons.

The arrangement is adapted to synchronize the charging of the first pair 122 of electrodes in relation to the second pair 126 of electrodes so that the second pair 126 of electrodes are negatively charged when the first pair 122 of electrodes are positively charged and vice versa.

The two transformers 124, 128 are of the same natural frequency and identical. By arranging the transformers 124, 128 adjacent each other in relative close proximity, their frequency cycles will become synced eventually in a steady state since they would influence each other during operation, due to Hertz and frequency laws. Accordingly, they can work with a synced frequency permanently. Accordingly, this synchronization happens spontaneously as soon as the transformers are turned on. According to an alternative, means may be provided to actively control the synchronization, such as arranging a one-way diode (a diode that synchronize the direction of the current in the same direction-the sinus or cosinus wave) in the path of each exit terminal.

Accordingly, each one of the transformers has two output terminals/poles, which are connected to the electrodes for charging the electrodes. When the potential reaches an amount that is sufficient for electric discharge, the above-mentioned phenomenon of electric discharge will take place. More specifically, a first electrode in the first pair 122 of electrodes and a first electrode in the second pair 126 of electrodes are connected to opposite terminals of a first transformer 124. Further, a second electrode in the first pair 122 of electrodes and a second electrode in the second pair 126 of electrodes are connected to opposite terminals of a second transformer 128.

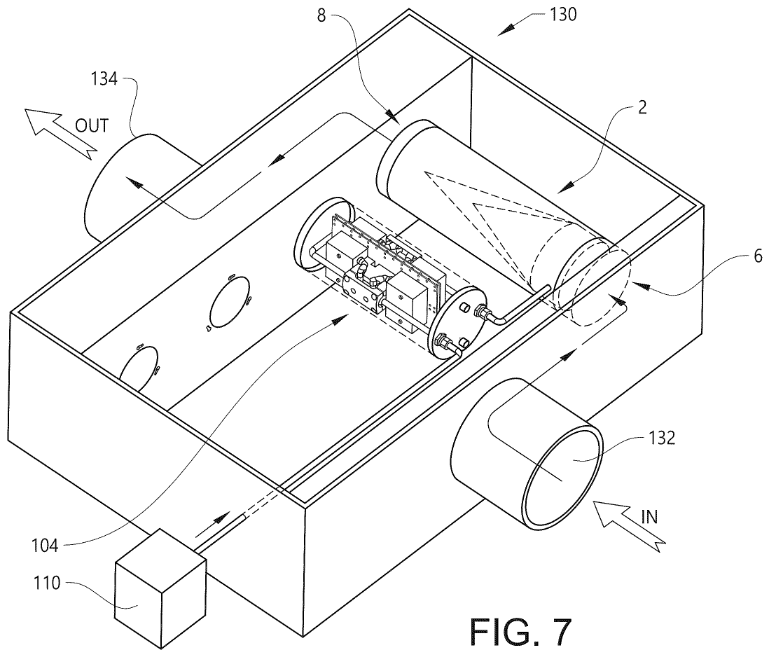

FIG. 7 is a perspective view of a mounting frame 130, wherein the hybrid filter unit 2 and the device for generating an oxidizing agent 104 according to FIG. 5 are indicated in a mounted state. The device for generating an oxidizing agent 104 and the hybrid filter unit 2 are fixedly connected to opposite walls of the mounting frame in a side-by-side relationship. The mounting frame 130 comprises an inlet 132 for inflow of air. The inlet 132 is in fluid communication with the inlet 6 of the hybrid filter unit 2. The mounting frame 130 comprises an outlet 134 for outflow of air. The outlet 134 is in fluid communication with the outlet 8 of the hybrid filter unit 2.

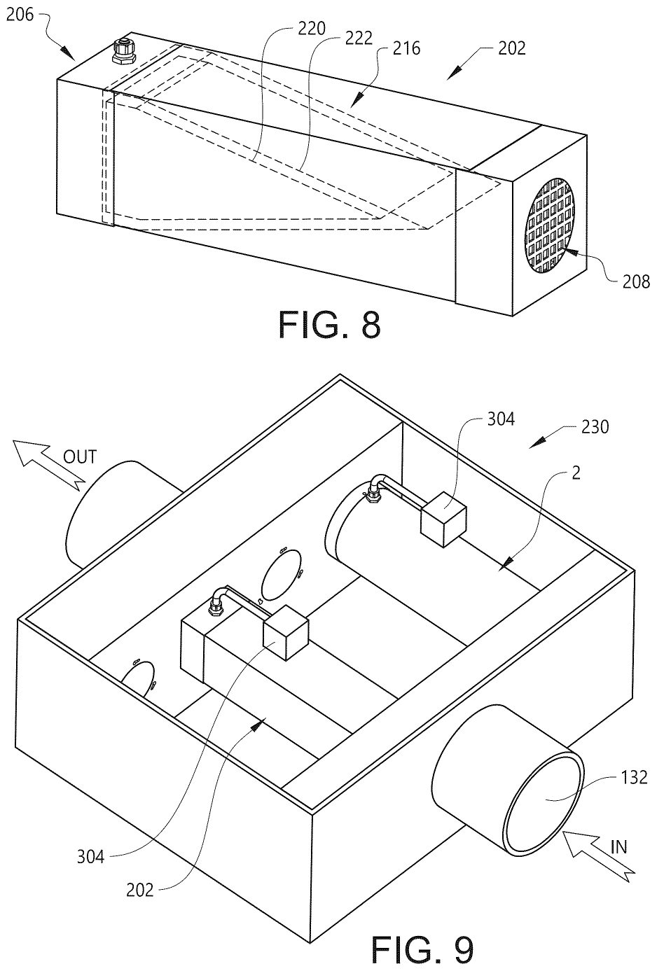

FIG. 8 is a schematic perspective view of a hybrid filter unit 202 according to a second embodiment. For ease of presentation, only the main differences in relation to the hybrid filter unit 2 according to the first embodiment will be described. The hybrid filter unit 202 comprises a housing that has a rectangular shape in cross section. The hybrid filter unit 202 further comprises a wall structure 216 arranged inside of the housing defining a mixing chamber for the fluid. The wall structure 216 has a through-flow area that at least partly decreases in a direction from an inlet 206 towards an outlet 208. More specifically, the wall structure 216 comprises a first wall 220 that defines the mixing chamber in a transverse direction of the housing. More specifically, the first wall 220 has a through-flow area that decreases in a direction from the inlet 206 towards the outlet 208. More specifically, the first wall 220 comprises two substantially flat rectangular portions, that are arranged in an inclined relationship so that they meet in a pointy end. Further, the first wall 220 comprises two spaced triangular-shaped side portions that are connected to the peripheries of the two substantially flat and inclined rectangular portions. The wall structure 216 further comprises a second wall 222 that has a similar shape as the first wall 220 and is arranged in the vicinity of the first wall 220 and spaced from the first wall 220 in an axial direction of the housing so that a gap is formed between the first wall 220 and the second wall 222. A wide end of each one of the first conical wall 220 and the second conical wall 222 faces the inlet 206.

FIG. 9 is a perspective view of a mounting frame, wherein two hybrid filter units 2, 202, each with an associated device 304 for generating an oxidizing agent, are indicated in a mounted state. The device 304 for generating an oxidizing agent may be operated according to a different principle in relation to the ionization by means of an electric discharge as described above. According to one example, the device 304 for generating an oxidizing agent may be comprise a light source, such as a UV light source, for radiating the fluid will UV light rays. Further, the oxidizing agent may be created from various types of sources such as stored in a capsule or generated in place by the means of a reactor or oxidant generator, etc.

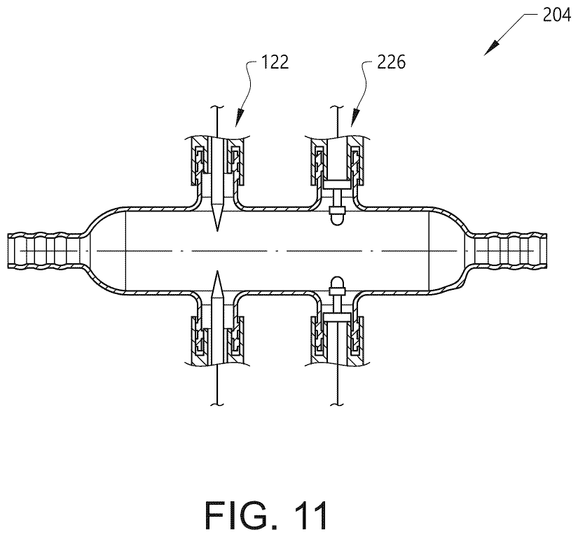

FIG. 11 is an alternative embodiment of an ionization unit 204 in relation to the ionization unit 104 shown in FIG. 10. For ease of presentation, only the main differences with the ionization unit will be described. The second electrode pair 126 in the ionization unit 104 is here replaced by a pair of light sources 226 such as UV lamps or UV-LEDs. Accordingly, each one of the light sources is arranged in one of the pipe-shaped portions of the container.

Alternatively, the UV light can be replaced with various light sources in different holes, each potentially facing an electrode and configured with a distinct polarity arrangement within the ionization container or chamber, tailored to the specific ionization target. Each electrode is capable of ionizing the surrounding fluid, emitting positrons and/or electrons through electric discharge, and working in conjunction with the UV light. This setup effectively eliminates the traditional heating, temperature fluctuations, and noise typically associated with corona discharge, whether the power source is AC or DC, this configuration is designed to generate a safe high voltage and efficiently target specific ionization elements in this embodiment.

It is to be understood that the present invention is not limited to the embodiments described above and illustrated in the drawings; rather, the skilled person will recognize that many changes and modifications may be made within the scope of the appended claims.

According to a further example, the internal walls structure/polarization unit may be different from cone-shaped. For example, it may have a pyramid-shape.

Further, according to the embodiment in FIG. 10, each one of the electrodes in the first pair of electrodes are charged so that they are simultaneously negatively or positively charged. In this way, a potential difference between each one of the electrodes and an environment of the respective electrode leads to that electric discharges take place from each one of the electrodes. Accordingly, a plurality of electric discharge structures formed at the same time from each one of the electrodes in the first pair. Similarly for the second pair of electrodes, they are charged so that electric discharges take place from each one of the electrodes. According to an alternative embodiment, a first one of the electrodes in one pair is positively charged and a second one of the electrodes in the same pair is simultaneously negatively charged, wherein continuous arc structures may be realized extending between the electrodes in each pair.

1. A hybrid filter unit, comprising.
a housing with an inlet for input of a fluid to be cleaned and an outlet for output of the fluid in a cleaned state;
a wall structure arranged inside of the housing defining a mixing chamber for the fluid, wherein the wall structure defines an inner cross section area that at least partly decreases in a direction from the inlet towards the outlet;
a nozzle arranged to provide an oxidizing agent to the mixing chamber so that the fluid may be subjected to the oxidizing agent and react with the oxidizing agent as it flows between the inlet and the outlet; and
a filtering structure arranged between an inner wall surface of the housing and an outer wall surface of the wall structure and wherein the filtering structure comprises volcanic Pumice wherein the wall structure is at least partly adapted for through-flow of the fluid from the mixing chamber to the filtering structure.

2. A hybrid filter unit according to claim 1, wherein the wall structure comprises a first wall and a second wall that are arranged in a spaced relationship, wherein an inner surface of the first wall defines the mixing chamber and wherein an outer surface of the second wall defines the outer wall surface of the wall structure.

3. A hybrid filter unit according to claim 2, wherein at least one of the first wall and the second wall has a wide end that is open to the mixing chamber and a pointy end opposite the wide end, wherein a cross section of the first wall and/or second wall is at least partly decreasing in a direction from the wide end to the pointy end.

4. A hybrid filter unit according to claim 3, wherein the wide end of said at least one of the first wall and the second wall faces towards the inlet for allowing entry of the fluid via the wide end into the mixing chamber.

5. A hybrid filter unit according to claim 3, wherein at least one of the first wall and the second wall is cone-shaped and wherein a base of the cone-shaped wall forms the wide end.

6. A hybrid filter unit according to claim 5, wherein a center axis of the cone-shaped first wall and/or a center axis of the second cone-shaped wall coincides with a center axis of the housing.

7. A hybrid filter unit according to claim 3, wherein the wide end of said at least one of the first wall and the second wall has a cross section size and shape substantially the same as or somewhat smaller than an inner cross section shape and size of the housing for receiving substantially a complete flow of the fluid flowing between the inlet and the outlet.

8. A hybrid filter unit according to claim 1, wherein the hybrid filter unit comprises an arrangement for attracting charged particles in the fluid flow, wherein the arrangement is arranged inside of the housing, wherein the arrangement for attracting charged particles comprises an inner electrically conducting element and an outer electrically conducting element arranged in a spaced relationship, and wherein the inner electrically conducting element and the outer electrically conducting element are adapted for being connected to a power source for being oppositely charged so that electrically charged particles in the fluid are attracted to one of the electrically conducting elements, wherein the power source is adapted to supply a low voltage and wherein it is connected so that the inner electrically conducting element is positively charged.

9. A hybrid filter unit according to claim 8, wherein the arrangement for attracting charged particles is arranged downstream of the nozzle in a direction of fluid flow between the inlet and the outlet.

10. A hybrid filter unit according to claim 2, wherein at least one of the inner electrically conducting element and the outer electrically conducting element forms one of said first wall and second wall of the wall structure defining the mixing chamber.

11. A hybrid filter unit according to claim 8, wherein the inner electrically conducting element is at least partly arranged inside of the outer electrically conducting element wherein the inner electrically conducting element and the outer electrically conducting element are arranged so that particles may be collected in a spacing between the inner electrically conducting element and the outer electrically conducting element.

12. A hybrid filter unit according to claim 8, wherein the outer electrically conducting element is arranged downstream of the inner electrically conducting element in a fluid flow direction.

13. A hybrid filter unit according to claim 1, wherein the hybrid filter unit comprises an element providing electrical insulation between the inner electrically conducting element and the outer electrically conducting element.

14. A hybrid filter unit according to claim 1, wherein the housing comprises a first open end, wherein the hybrid filter unit comprises a first cap that defines an opening and is adapted to be connected to the first open end of the housing in a way that the cap opening forms the inlet of the housing, wherein at least one wall in the wall structure is attached to the cap.

15. A hybrid filter unit according to claim 13, wherein at least one of the inner electrically conducting element and the outer electrically conducting element is releasably attached to the first cap for allowing access to a spacing between the inner electrically conducting element and the outer electrically conducting element for cleaning.

16. A hybrid filter unit according to claim 14, wherein the first cap forms the electrically insulating element between the inner electrically conducting element and the outer electrically conducting element

17. A hybrid filter unit according to claim 14, wherein the housing comprises a second open end, wherein the hybrid filter unit comprises a second cap that defines an opening and is adapted to be connected to the second open end of the housing in a way that the cap opening forms the outlet of the housing.

18. A hybrid filter unit according to any preceding claim, wherein the nozzle is arranged in a wall of the housing so that its inner end is located inside of the housing and a body of the nozzle extends transversally relative to a center axis of the housing between the inlet and the outlet.

19. A hybrid filter unit according to claim 18, wherein the nozzle inner end is arranged inside of the mixing chamber defined by the wall structure.

20-23. (canceled)

24. A fluid cleaning arrangement comprising a device for generating an oxidizing agent, and a hybrid filter unit according to claim 1, wherein the oxidizing agent generating device is arranged separate from the hybrid filter unit and an outlet of the oxidizing agent generating device is operatively connected to the nozzle for providing the oxidizing agent to the mixing chamber of the hybrid filter unit.

25-30. (canceled)
